SEQUENCE LISTING (3 sequences)

SEQ ID NO: 1 - DNA, 15 bp, Artificial Sequence
  KOZAK
ctgcagcccaccacc

SEQ ID NO: 2 - DNA, 37 bp, Artificial Sequence
  Primer 1
atacgcaaacggatcgggcctatgagttccatcttag

SEQ ID NO: 3 - DNA, 35 bp, Artificial Sequence
  Primer 2
ggtggtgggctgcagctgggcgcggaaagaggacg

(19) United States
(12) Patent Application Publication
(10) Pub. No.: US 2022/0265751 A1
(43) Pub. Date: Aug. 25, 2022

(54) COMPOSITION FOR INHIBITING NERVE CELL INJURY CAUSED BY Aß

(71) Applicant: SUMITOMO CHEMICAL COMPANY, LIMITED, Tokyo (JP)

(72) Inventors: Katsunori SASAKI, Tokyo (JP); Kumiko KOBAYASHI, Osaka-shi (JP); Kiyoshi HIGASHI, Osaka-shi (JP)

(73) Assignee: SUMITOMO CHEMICAL COMPANY, LIMITED, Tokyo (JP)

(21) Appl. No.: 17/632,454

(22) PCT Filed: Aug. 7, 2020
(86) PCT No.: PCT/JP2020/030292

§ 371 Date: Feb. 2, 2022

(30) Foreign Application Priority Data
Aug. 7, 2019 (JP) 2019-145563

(51) Int. Cl. A61K 36/484; A61K 36/906; A61K 36/899; A61K 36/725; A61K 36/16; A61P 25/28

(52) U.S. Cl. CPC A61K 36/484; A61K 36/906; A61K 36/899; A61K 36/725; A61K 36/16; A61P 25/28

(57) ABSTRACT

The problem to be solved by this invention is to provide a composition for inhibiting nerve cell injury caused by Aβ. This problem is solved by a composition for inhibiting nerve cell injury caused by Aβ, comprising a Kampo extract that contains a glycyrrhiza extract.

### TECHNICAL FIELD

The present invention relates to, for example, a composition for inhibiting nerve cell injury caused by Aβ.

### BACKGROUND ART

In neurodegenerative diseases, such as Alzheimer's disease, amyloid fibrils (Aβ oligomers) formed by oligomerization of amyloid β peptides (Aβ) are detected in the cerebrum and hippocampus of diseased patients. Accumulation of Aβ oligomers in the brain is believed to be deeply involved in the onset of neurodegenerative diseases, and it has been demonstrated that the amount of Aβ oligomers in the brain determined based on Aβ-imaging is inversely correlated with the score on the Mini-Mental State Examination used for the diagnosis of Alzheimer's disease (Non-patent Literature (NPL) 1)).

In terms of the association of Aβ oligomers with neurodegenerative diseases, it has been reported that amyloid fibril accumulation was observed in animals infused with Aβ oligomers into the cerebral ventricles, showing human Alzheimer's disease-like symptoms, such as cognitive and learning disabilities, and that suppression of the amyloid fibril accumulation ameliorated the Alzheimer's disease-like symptoms (NPL 2).

In terms of the association of Aβ oligomers with nerve cells, Aβ oligomers have been demonstrated to damage nerve cells in an experimental system using cultured nerve cells from non-human mammals.

These findings indicate that inhibition of the formation of Aβ oligomers and inhibition of nerve cell injury caused by Aβ oligomers can prevent the onset of many neurodegenerative diseases, including Alzheimer's disease.

In Alzheimer's disease, neurite degeneration is one of the most characteristic neuropathological changes. In mice, it has been known that the amount of microtubule-associated protein 2 (MAP-2) specifically expressed in nerve cells decreases as neurites degenerate (NPL 3). Further, it has been known that MAP-2 begins to be expressed when differentiation of neural precursor cells into mature nerve cells begins, and that MAP-2 in mature nerve cells is localized almost specifically in dendrites. That is, MAP-2 can be used as an indicator of the number of viable nerve cells with extended dendrites. It has been reported so far that the amount of MAP-2 decreases simultaneously with the accumulation of Aβ oligomers in the nerve cells in model mice with Alzheimer's disease, and that the addition of Aβ oligomers decreases the amount of MAP-2 in cultured mouse nerve cells (NPL 4 and NPL 5).

### CITATION LIST

### Non-Patent Literature

NPL 1: Ng, S., Villemagne, V. L., Berlangieri, S., Lee, S. T., Cherk, M., Gong, S. J., Ackermann, U., Saunder, T., Tochon-Danguy, H., Jones, G., Smith, C., O'Keefe, G., Masters, C. L. and Rowe, C. C. (2007), Visual assessment versus quantitative assessment of 11C-PIB PET and 18F-FDG PET for detection of Alzheimer's disease, The Journal of Nuclear Medicine 48, pp. 547-52
NPL 2: Walsh, D. M., Klyubin, I., Fadeeva, J. V., Cullen, W. K., Anwyl, R., Wolfe, M. S., Rowan, M. J. and Selkoe, D. J. (2002), Naturally secreted oligomers of amyloid beta protein potently inhibit hippocampal long-term potentiation in vivo., Nature 416, pp. 535-39
NPL 3: Adlard, P. A. and Vickers, J. C., (2002), Morphological distinct plaque types differentially affect dendritistic structure and organization in the early and late stages of Alzheimer's disease, Acta Neuropathologica 103, pp. 377-83
NPL 4: Takahashi, R. H., Capetillo-Zarate, E., Lin, M. T., Milner, T. A. and Gouras, G. K. (2013), Accumulation of intraneuronal beta-amyloid 42 peptides is associated with early changes in microtubule-associated protein 2 in neurites and synapses, PLoS ONE 8, e51965
NPL 5: Fifre, A., Sponne, I., Koziel, V., Kriem, B., Yen Potin, F. T., Bihain, B. E., Olivier, J. L., Oster, T. and Pillot, T. (2006). Microtubule-associated protein MAP1A, MAP1B, and MAP2 proteolysis during soluble amyloid beta-peptide-induced apoptosis, Synergistic involvement of calpain and caspase-3, The Journal of Biological Chemistry 281, pp. 229-40

### SUMMARY OF INVENTION

### Technical Problem

An object of the present invention is to provide a composition for inhibiting nerve cell injury caused by Aβ.

### Solution to Problem

The present inventors conducted extensive research to solve the above problem and consequently found that Kampo extracts that contain a glycyrrhiza extract can inhibit nerve cell injury caused by Aβ. Based on these findings, the present inventors conducted further research. Accordingly, the present invention has been accomplished.

More specifically, the present invention encompasses the following embodiments.

Item 1. A composition for inhibiting nerve cell injury caused by Aβ, comprising a Kampo extract that contains a glycyrrhiza extract.

Item 2. The composition according to Item 1, wherein the Kampo extract does not contain a crude drug extract other than a glycyrrhiza extract, or further contains at least one crude drug extract selected from the group consisting of a processed ginger extract, a jujube extract, a wheat extract, a ginger extract, and a platycodon root extract.

Item 3. The composition according to Item 1 or 2, wherein the Kampo extract further contains at least one crude drug extract selected from the group consisting of a processed ginger extract, a jujube extract, and a wheat extract.

Item 4. The composition according to any one of Items 1 to 3, wherein the Kampo extract is at least one member selected from the group consisting of a kanzokankyoto extract and a kanbakutaisoto extract.

Item 5. The composition according to Item 4, wherein the mass ratio of crude drug raw materials for use in obtaining the kanzokankyoto extract is such that glycyrrhiza to processed ginger is 1:0.1 to 1.5.

Item 6. The composition according to Item 4 or 5, wherein the mass ratio of crude drug raw materials for use in obtaining the kanzokankyoto extract is such that glycyrrhiza to processed ginger is 1:0.2 to 1.0.

Item 7. The composition according to Item 4, wherein the mass ratio of crude drug raw materials for use in obtaining the kanbakutaisoto extract is such that glycyrrhiza to jujube to wheat is 1:0.5 to 2.0:2.6 to 6.6.

Item 8. The composition according to Item 4 or 7, wherein the mass ratio of crude drug raw materials for use in obtaining the kanbakutaisoto extract is such that glycyrrhiza to jujube to wheat is 1:0.7 to 1.7:3.5 to 4.5.

Item 9. The composition according to any one of Items 1 to 8, which comprises the Kampo extract in an amount such that the daily application amount is 0.1 to 15 g.

Item 10. The composition according to any one of Items 1 to 9, which comprises the Kampo extract in an amount such that the daily application amount is 0.2 to 4.5 g.

Item 11. The composition according to any one of Items 1 to 10 for use in the prevention, amelioration, or treatment of diseases or symptoms in which nerve cell injury caused by Aβ is observed.

Item 12. The composition according to Item 11, wherein the disease or symptom is Alzheimer's disease, mild cognitive impairment, Lewy body dementia, Down syndrome, Dutch hereditary cerebral hemorrhage with amyloidosis, Guam parkinsonism-dementia complex, cerebral amyloid angiopathy, inclusion body myositis, frontotemporal dementia, age-related macular degeneration, or Pick's disease.

Item 13. The composition according to any one of Items 1 to 12, which is a food composition.

The present invention also encompasses the following embodiments.

Item 14. A composition comprising a Kampo extract that contains a glycyrrhiza extract, for use in a method for inhibiting nerve cell injury caused by Ap.

Item 15. A method for inhibiting nerve cell injury caused by Aβ, comprising applying (e.g., ingesting, inoculating, or administering) a composition comprising a Kampo extract that contains a glycyrrhiza extract to a subject in need of inhibiting nerve cell injury caused by Aβ.

Item 16. Use of a Kampo extract that contains a glycyrrhiza extract, in the manufacture of a composition for inhibiting nerve cell injury caused by Aβ.

Item 17. Use of a composition comprising a Kampo extract that contains a glycyrrhiza extract, for inhibiting nerve cell injury caused by Aβ.

### Advantageous Effects of Invention

The present invention can provide a composition for inhibiting nerve cell injury caused by Aβ.

### DESCRIPTION OF EMBODIMENTS

In the present specification, the expressions “comprise,” “contain”, and “include” encompass the concepts of containing, including, consisting essentially of, and consisting of.

In one embodiment, the present invention relates to a composition for inhibiting nerve cell injury caused by Aβ, comprising a Kampo extract that contains a glycyrrhiza extract (which may be referred in the present specification as “the composition of the present invention”). Details are described below.

### 1. Components

Glycyrrhiza extract is a type of crude drug extract and may be any glycyrrhiza extract as long as it is an extract of glycyrrhiza as a crude drug. The plant raw material of glycyrrhiza is not particularly limited. Typical examples include plants of the genus Glycyrrhiza, such as Glycyrrhiza uralensis Fischer, Glycyrrhiza glabra Linné, and Glycyrrhiza inflata. For glycyrrhiza, any part of the plant raw materials may be used as long as it can be used as a crude drug, and typically includes the roots and stolons of the plant raw materials. The state of the glycyrrhiza is not particularly limited and is typically in a dry state. Extraction can be performed by using a method commonly used to extract a crude drug extract. Specific examples include a method of extracting glycyrrhiza at an ordinary temperature to a high temperature by using water or hydrous ethanol as an extraction solvent. The crude drug extract that contains a glycyrrhiza extract may be, for example, an extract, a diluted extract, or a concentrated extract (including liquid, semi-liquid, and solid forms). The glycyrrhiza extracts may be used singly or in a combination of two or more.

The Kampo extract may be any physiologically acceptable Kampo extract. The types and content ratios of crude drug extracts constituting the Kampo extract are also not particularly limited. For example, crude drug extracts that can be used for food products and crude drug extracts that can be used for pharmaceuticals may be widely used. The Kampo extract is a composition consisting of a single crude drug extract, a composition containing a single crude drug extract, or a composition containing two or more different crude drug extracts. For example, the Kampo extract may be an extract, a diluted extract, or a concentrated extract (including liquid, semi-liquid, and solid forms). Of Kampo extracts, the Kampo extract used in the present invention contains a glycyrrhiza extract. Examples of the Kampo extract that contains a glycyrrhiza extract include kanzokankyoto extract, kanbakutaisoto extract, kanzoto extract, kikyoto extract, anchusan extract, ireito extract, ogikenchuto extract, orento extract, otsujito extract, kakkonto extract, kakkontokasenkyushin'i extract, kanzoshashinto extract, kigikenchuto extract, kyukikyogaito extract, kyoseihatekigan extract, kyososan extract, kufugedokusan(to) extract, keigairengyoto extract, keishikakakkonto extract, keishikashakuyakuto extract, keishikashakuyakudaioto extract, keishito extract, keishininjinto extract, keimakakuhanto extract, kenchuto extract, koshaheiisan extract, koshayoisan extract, kosharikkunshito extract, kososan extract, gokoto extract, goshakusan extract, saikanto extract, saikokeishito extract, saikoseikanto extract, saishakurikkunshito extract, jiinkokato extract, jiinshihoto extract, shigyakusan extract, shikunshito extract, jidabokuippo extract, shakanzoto extract, shakuyakukanzoto extract, jumihaidokuto extract, junchoto extract, shokenchuto extract, shosaikoto extract, shoseiryuto extract, shomakakkonto extract, jingyokyokatsuto extract, jingyobofuto extract, seihaito extract, soshikokito extract, daiokanzoto extract, chikujountanto extract, choijokito extract, tokakujokito extract, tokikenchuto extract, tokishigyakuto extract, tokishigyakukagoshuyushokyoto extract, dokkatsukakkonto extract, nijutsuto extract, ninjinto extract, hainoto extract, bakumondoto extract, hangeshoshinto extract, heiisan extract, kamiheiisan extract, hochuekkito extract, maoto extract, makyokansekito extract, makyoyokukanto extract, yokuininto extract, yokukansan extract, rikkunshito extract, ryokyojutsukanto extract, and ryokeijutsukanto extract.

In one embodiment of the present invention, without particular limitation, the Kampo extract preferably does not contain a crude drug extract other than a glycyrrhiza extract, or further contains at least one crude drug extract selected from the group consisting of a processed ginger extract, a jujube extract, a wheat extract, a ginger extract, and a platycodon root extract, more preferably further contains at least one crude drug extract selected from the group consisting of a processed ginger extract, a jujube extract, and a wheat extract, and still more preferably further contains a processed ginger extract, from the standpoint of, for example, inhibiting nerve cell injury caused by Aβ. Without particular limitation, such Kampo extract is preferably, for example, a kanzokankyoto extract, a kanbakutaisoto extract, a kanzoto extract, a kikyoto extract, and the like, more preferably a kanzokankyoto extract, a kanbakutaisoto extract, and the like, and still more preferably a kanzokankyoto extract and the like, from the standpoint of inhibiting nerve cell injury caused by Aβ. When the Kampo extract further contains a crude drug extract other than and in addition to a glycyrrhiza extract, the Kampo extract may be, but is not particularly limited to, for example, a mixture of crude drug extracts, or may be an extract of a mixture of crude drug raw materials.

Ginger extract may be any ginger extract as long as it is an extract of ginger as a crude drug. The plant raw material of ginger is not particularly limited. Typical examples include plants of the genus Zingiber, such as Zingiber officinale Roscoe. For ginger, any part of the plant raw materials may be used as long as it can be used as a crude drug, and typically includes the rhizomes of the plant raw materials. The state of the ginger is not particularly limited and is a dry state or a non-dry state. Extraction can be performed by using a method commonly used to extract a crude drug extract. Specific examples include a method of extracting ginger at an ordinary temperature to a high temperature by using water or hydrous ethanol as an extraction solvent. The ginger extracts may be used singly or in a combination of two or more.

Processed ginger extract may be any processed ginger extract as long as it is an extract of processed ginger as a crude drug. The plant raw material of processed ginger is not particularly limited. Typical examples include plants of the genus Zingiber, such as Zingiber officinale Roscoe. For processed ginger, any part of the plant raw materials may be used as long as it can be used as a crude drug, and typically includes the rhizomes of the plant raw materials. The state of the processed ginger is not particularly limited and is typically in a dry state. Extraction can be performed by using a method commonly used to extract a crude drug extract. Specific examples include a method of extracting processed ginger at an ordinary temperature to a high temperature by using water or hydrous ethanol as an extraction solvent. The processed ginger extracts may be used singly or in a combination of two or more.

Jujube extract may be any jujube extract as long as it is an extract of jujube as a crude drug. The plant raw material of jujube is not particularly limited. Typical examples include plants of the genus Ziziphus, such as Zizyphus jujuba Miller var. inermis Rehder. For jujube, any part of the plant raw materials may be used as long as it can be used as a crude drug, and typically includes the fruit of the plant raw materials. The state of jujube is not particularly limited and is typically in a dry state. Extraction can be performed by using a method commonly used to extract a crude drug extract. Specific examples include a method of extracting jujube at an ordinary temperature to a high temperature by using water or hydrous ethanol as an extraction solvent. The jujube extracts may be used singly or in a combination of two or more.

Wheat extract may be any wheat extract as long as it is an extract of wheat as a crude drug. The plant raw material of wheat is not particularly limited. Typical examples include plants of the genus Triticum, such as Triticum aestivum. For wheat, any part of the plant raw materials may be used as long as it can be used as a crude drug, and typically includes the fruit of the plant raw materials. The state of the wheat is not particularly limited and is typically in a dry state. Extraction can be performed by using a method commonly used to extract a crude drug extract. Specific examples include a method of extracting wheat at an ordinary temperature to a high temperature by using water or hydrous ethanol as an extraction solvent. The wheat extracts may be used singly or in a combination of two or more.

Platycodon root extract may be any platycodon root extract as long as it is an extract of platycodon root as a crude drug. For platycodon root, any part of the plant raw materials may be used as long as it can be used as a crude drug, and typically includes plants of the genus Platycodon, such as Platycodon glandiflorum (Jacq.) A. DC. For platycodon root, any part of the plant raw materials may be used as long as it can be used as a crude drug, and typically includes the roots, or the roots excluding the cork skin, of the plant raw materials. The state of the platycodon root is not particularly limited and is typically in a dry state. Extraction can be performed by using a method commonly used to extract a crude drug extract. Specific examples include a method of extracting platycodon root at an ordinary temperature to a high temperature by using water or hydrous ethanol as an extraction solvent. The platycodon root extracts may be used singly or in a combination of two or more.

The mass ratio of the crude drug raw materials for use in obtaining Kampo extracts is not particularly limited, and a mass ratio according to known mass ratios may be used for each Kampo extract. The mass ratio of the crude drug raw materials for use in obtaining a kanzokankyoto extract is not particularly limited and is, for example, such that glycyrrhiza to processed ginger is preferably 1:0.1 to 1.5, and more preferably 1:0.2 to 1.0. As another example, the mass ratio of the crude drug raw materials for use in obtaining a kanbakutaisoto extract is not particularly limited and is, for example, such that glycyrrhiza to jujube to wheat is preferably 1:0.5 to 2.0:2.6 to 6.6, and more preferably 1:0.7 to 1.7:3.5 to 4.5. As another example, the mass ratio of the crude drug raw materials for use in obtaining a kikyoto extract is not particularly limited and is, for example, such that glycyrrhiza to platycodon root is preferably 1:0.1 to 2.0, and more preferably 1:0.1 to 1.2.

The Kampo extracts may be used singly or in a combination of two or more.

### 2. Use

The Kampo extract that contains a glycyrrhiza extract is capable of inhibiting the formation of Aβ oligomers and also inhibiting an AB-induced decrease in the MAP-2 promoter activity. Therefore, the Kampo extract is expected to be used as a component of, for example, a composition for inhibiting nerve cell injury caused by Aβ, a composition for inhibiting the formation of Aβ oligomers, and a composition for inhibiting an Aβ-induced decrease in the MAP-2 promoter activity. The Kampo extract exhibiting the above effects can be used for prevention, amelioration, or treatment of diseases or symptoms in which nerve cell injury caused by Aβ is observed. Examples of such diseases and symptoms include neurodegenerative diseases (or neurodegenerative conditions). More specific examples include Alzheimer's disease, mild cognitive impairment, Lewy body dementia, Down syndrome, Dutch hereditary cerebral hemorrhage with amyloidosis, Guam parkinsonism-dementia complex, cerebral amyloid angiopathy, inclusion body myositis, frontotemporal dementia, age-related macular degeneration, and Pick's disease.

“Aβ” refers to amyloid β-proteins and is a general term that encompasses multiple molecular species. A polymer consisting of two or more Aβ molecules is referred to as an “Aβoligomer.” Examples of Aβ include hydrophobic peptides Aβ₁₋₄₂ (molecular weight: 4,514) and Aβ₁₋₄₀ (molecular weight: 4,329.8). Of these, in particular, Aβ₁₋₄₂ oligomers are believed to have a relatively large impact on nerve cell injury. The Aβ oligomer may be, but is not particularly limited to, for example, an AB dimer to a 50-mer Aβ oligomer, or an Aβ dimer to a 30-mer Aβ oligomer. Without particular limitation, the Aβoligomer may be formed spontaneously, for example, by allowing it to stand at room temperature or 37° C. in a solvent, such as a culture medium or a phosphate buffer solution.

A nerve cell (neuron) is divided into a cell body with a cell nucleus, dendrites that receive a signal from other cells, and an axon that sends out a signal to other cells, and plays an important role in transmitting signals via the dendrites and the axon. The term “nerve cell injury” in the present specification refers to a decrease in the amounts of MAP-2 genes and proteins present in dendrites, and further refers to the potential of impairment in signaling functions.

In one embodiment, the composition of the present invention can be for therapeutic or non-therapeutic use (prevention, amelioration, etc.). The composition of the present invention can be applied (ingested, inoculated, administered, etc.) to a subject, for example, mammals, including humans. (Examples of the mammals include, but are not particularly limited to humans, monkeys, mice, rats, rabbits, dogs, cats, horses, cows, and pigs, with humans being preferred.)

The content of the Kampo extract in the composition of the present invention is not particularly limited as long as nerve cell injury caused by Aβ can be inhibited. Although the content is not particularly limited, the daily application amount is preferably, for example, 0.1 to 15 g, and more preferably 0.2 to 4.5 g. When the Kampo extract is a kanzokankyoto extract, the daily amount is more specifically as follows, without particular limitation. The daily amount of the kanzokankyoto extract is not particularly limited and is preferably, for example, 0.1 to 10 g of an extract obtained by extracting 0.5 to 10 g of glycyrrhiza and 0.2 to 5 g of processed ginger as crude drugs, more preferably 0.2 to 5 g of an extract obtained by extracting 0.5 to 4 g of glycyrrhiza and 0.2 to 2 g of processed ginger, and still more preferably 0.2 to 2 g of an extract obtained by extracting 0.5 to 2 g of glycyrrhiza and 0.2 to 1 g of processed ginger. When the Kampo extract is a kanbakutaisoto extract, the daily amount is more specifically as follows, without particular limitation. The daily amount of the kanbakutaisoto extract is not particularly limited and is preferably, for example, 0.1 to 15 g of an extract obtained by extracting 0.5 to 10 g of glycyrrhiza, 0.6 to 12 g of jujube, and 2 to 40 g of wheat as crude drugs, more preferably, for example, 0.5 to 10 g of an extract obtained by extracting 0.5 to 5 g of glycyrrhiza, 0.6 to 6 g of jujube, and 2 to 20 g of wheat, and still more preferably, for example, 1 to 4.5 g of an extract obtained by extracting 1 to 2 g of glycyrrhiza, 1.2 to 2.4 g of jujube, and 4 to 8 g of wheat. When the Kampo extract is a kanzoto extract, the daily amount is more specifically as follows, without particular limitation. The daily amount of the kanzoto extract is not particularly limited and is preferably, for example, 0.1 to 6 g of an extract obtained by extracting 0.1 to 16 g of glycyrrhiza as a crude drug, more preferably, for example, 0.1 to 3 g of an extract obtained by extracting 0.2 to 8 g of glycyrrhiza, and still more preferably, for example, 0.2 to 0.7 g of an extract obtained by extracting 0.5 to 2 g of glycyrrhiza. When the Kampo extract is a kikyoto extract, the daily amount is more specifically as follows, without particular limitation. The daily amount of kikyoto extract is not particularly limited and is preferably, for example, 0.1 to 6 g of an extract obtained by extracting 0.1 to 16 g of glycyrrhiza and 0.1 to 8 g of platycodon root as crude drugs, more preferably, for example, 0.1 to 3 g of an extract obtained by extracting 0.2 to 8 g of glycyrrhiza and 0.2 to 4 g of platycodon root, and still more preferably, for example, 0.2 to 1 g of an extract obtained by extracting 0.3 to 3 g of glycyrrhiza and 0.2 to 2 g of platycodon root.

The composition of the present invention may consist only of the Kampo extract that contains a glycyrrhiza extract, or may contain additional components in addition to the Kampo extract that contains a glycyrrhiza extract. The amount of the Kampo extract that contains a glycyrrhiza extract contained in the composition of the present invention can be appropriately set according to the form, dosage form, symptoms, and the like. When additional components are contained, the proportion of the Kampo extract that contains a glycyrrhiza extract in the composition of the present invention is not particularly limited and is typically 0.1 to 99.9 mass %. For example, when the Kampo extract is a kanbakutaisoto extract, the proportion is not particularly limited, and is preferably 1 to 42 mass %, and more preferably 3 to 30 mass %. As another example, when the Kampo extract is a kanzokankyoto extract, the proportion is not particularly limited, and is preferably 0.3 to 50 mass %, and more preferably 0.5 to 15 mass %. As another example, when the Kampo extract is a kanzoto extract, the proportion is not particularly limited, and is preferably 0.3 to 31 mass %, and more preferably 0.5 to 22 mass %. As another example, when the Kampo extract is a kikyoto extract, the proportion is not particularly limited, and is preferably 3 to 32 mass %, and more preferably 9.5 to 16 mass %.

The composition of the present invention may be in any pharmaceutical form, such as jelly, powder, granule, tablet, capsule, liquid, suspension, or emulsion form.

The composition of the present invention is preferably applied, for example, orally, without particular limitation. Application methods other than oral application can also be used. For example, a base may be incorporated in the composition of the present invention for the purpose of efficient in vivo transportation. The base may be a general-purpose gelling agent. Since appropriate storage stability is required, a gelling agent other than gelatin, for example, is preferred, without particular limitation. Preferable examples of the gelling agent include, but are not particularly limited to, carrageenan, locust bean gum (carob bean gum), xanthan gum, polyacrylic acid, gellan gum, psyllium seed gum, tara gum, guar gum, agar, pectin, and alginic acid.

The composition of the present invention can be used as food products, pharmaceuticals, quasi-drugs, feed, and the like. The composition of the present invention also encompasses the meanings of additives of food products, pharmaceuticals, quasi-drugs, feed, and the like.

The term “food product” used in the present specification also encompasses the meanings of food products and beverages for the purpose of health, health maintenance, health promotion, etc. (e.g., health food, functional food, dietary supplements, supplements, food for specified health uses, food with nutrient function, or food with functional claims), food for infants, food for pregnant and nursing women, food for the sick, and the like.

The food products include any food products and beverages that can be consumed by animals (including humans). The type of food products is not particularly limited. Examples include dairy products; fermented food products (e.g., yogurt and cheese); beverages (e.g., soft beverages such as coffee, juice, and tea beverages, carbonated beverages, milk beverages, lactic acid bacteria beverages, lactic acid bacteria-containing beverages, yogurt beverages, and liquors such as Japanese sake, fruit wine, and western liquors); spreads (e.g., custard cream); pastes (e.g., fruit pastes); western confectioneries (e.g., donuts, pies, cream puffs, gum, candies, jellies, cookies, cakes, chocolates, and puddings); Japanese confectioneries (e.g., daifuku (mochi stuffed with sweet filling), mochi (rice cake), manju (flour-based pastry stuffed with sweet filling), castella (sponge cake), anmitsu (Japanese sweet made with sweet red bean paste and agar cubes), and yokan (azuki bean jelly)); frozen desserts (e.g., ice cream, ice pops, and sorbet); food products (e.g., curry, gyudon (beef-on-rice bowl), zosui (seasoned rice porridge containing other ingredients), miso soup, soup, meat sauce, pasta, pickles, and jam); and seasonings (e.g., dressing, umami seasonings, furikake (sprinkled flavorings for rice), and soup stock). The method for producing food products is also not particularly limited, and a known method may be appropriately used.

The dosage unit form when the food product is used as a supplement is not particularly limited and may be appropriately selected. Examples include tablets, capsules, granules, liquids, powders, and jellies.

The amount of intake of the food product may be appropriately determined according to various conditions such as the weight, age, gender, and symptoms of the subjects who are to consume the food product. The daily intake amount of the Kampo extract may be, for example, the application amount mentioned above.

To prepare pharmaceuticals and quasi-drugs, the Kampo extract may be used as is. Alternatively, the Kampo extract may be used with a non-toxic carrier, diluent, or excipient acceptable in pharmaceuticals to obtain preparations for pharmaceutical use in the form of, for example, tablets (e.g., uncoated tablets, sugar-coated tablets, film-coated tablets, effervescent tablets, chewable tablets, and lozenges), capsules, pills, powders (dispersants), fine granules, granules, liquids, suspensions, emulsions, jellies, syrups, and pastes. Examples of non-toxic carriers acceptable in pharmaceuticals include binders, additives, flavors, buffers, thickeners, colorants, stabilizers, emulsifiers, dispersants, suspending agents, and preservatives.

The administration method for the pharmaceuticals and quasi-drugs is not particularly limited. Examples include oral administration, rectal administration, enteral administration, buccal administration, intra-arterial administration, intravenous administration, and transdermal administration.

The administration amount of the pharmaceuticals and quasi-drugs may be appropriately determined according to various conditions such as the body weight, age, gender, and symptoms of the patient. The daily administration amount of the Kampo extract may be, for example, the application amount mentioned above.

Several Kampo extracts are already available for the market as pharmaceutical products. However, from the standpoint of availability, the composition of the present invention according to one embodiment is preferably, for example, used as a food product, without particular limitation.

The food products, pharmaceuticals, quasi-drugs, etc. may be provided with a label stating, for example, that the product is for preventing the onset or promoting the delay of the onset of neurodegenerative diseases, such as Alzheimer's disease, or for inhibiting deterioration in cognitive function.

In one embodiment, the composition of the present invention may be used in combination with other compositions. For example, a combined use of the composition of the present invention with materials and composition that are expected to prevent the onset or promote the delay of the onset of neurodegenerative diseases, such as Alzheimer's disease, can further improve the effect of preventing neurodegenerative diseases, such as Alzheimer's disease and promoting the delay of the onset of the diseases.

In one embodiment, the composition of the present invention is preferably, for example, continuously ingested, without particular limitation. For example, the ingestion is preferably performed continuously for 1 week or more, more preferably for 2 weeks or more, and still more preferably 3 weeks or more.

In one embodiment, the composition of the present invention may comprise the Kampo extract in a daily application amount effective to inhibit nerve cell injury caused by Aβ. In this case, the composition of the present invention may be packaged so as to provide the daily effective application amount. The packaging form may be a single package or multiple packages as long as the effective daily application amount can be provided. When the effective daily application amount is divided into multiple packages, the multiple packages containing the effective daily application amount may be provided as a set.

The packaging form is not particularly limited as long as a predetermined amount can be contained. Examples include bags, wrapping paper, paper containers, soft packs, cans, bottles, and capsules.

### EXAMPLES

The present invention is described in detail below with reference to Examples. However, the present invention is not limited to these Examples.

### Example 1: Inhibition Test Against Nerve Cell Injury in Mice Caused by Aβ

a) Preparation of Mouse ES Stable Transformant Cell Line Containing a Fusion Gene of MAP-2 Promoter Gene with Green Luciferase (SLG) Gene

Mouse ES cells were obtained according to the method disclosed in non-patent literature (Le Coz, F. et al., J. Toxicol. Sci. 40, 251-261 (2015)). The plasmid vector pGL4.17 (produced by Promega Corporation, catalog number: E6721) was cleaved with restriction enzymes EcoRV and BamHI, and the 5 kb promoter gene upstream of the mouse MAP-2 gene and the SLG gene having a KOZAK sequence (ctgcagcccaccacc (SEQ ID NO: 1)) were connected according to an In-Fusion method. The 5 kb promoter sequence upstream of the mouse MAP-2 gene was amplified by the PCR method using primers (5′-atacgcaaacggatcgggcctatgagttccatcttag-3′ (SEQ ID NO: 2) and 5′-ggtggtgggctgcagctgggcgcggaaagaggacg-3′ (SEQ ID NO: 3)) and using the genomic DNA of the mouse ES cells as a template. The SLG gene having a KOZAK sequence was artificially synthesized in Invitrogen. After each fragment was connected according to an In-Fusion method, the sequences of the MAP-2 promoter gene and the SLG gene were confirmed using a DNA sequencer, and the resulting product was named a “MAP-2-Luc plasmid.”

Next, the MAP-2-Luc plasmid was reacted with a restriction enzyme SalI for linearization, and the plasmid purified on agarose gel was introduced into the mouse ES cells using lipofectamine 2000 (produced by Thermo Fisher Scientific, catalog number: 11668089). The transgenic mouse ES cells were treated with trypsin to obtain single cells, which were dispersed in a medium containing 100-150 μg/ml G418 (produced by Thermo Fisher Scientific, catalog number: 11811031) and seeded on mouse embryonic fibroblasts (produced by ReproCELL Inc., catalog number: RCHEFC003) so that each well contained a single cell in a 96-well plate. After passage several times, 79 cell lines of the remaining cells were obtained as drug-resistant cell lines. Each cell line was differentiated into nerve cells as described below, and luciferase activity (fluorescence value) on day 0 and day 11 of differentiation induction was measured using a Triple Luciferase Assay System (produced by Promega Corporation). The clones that showed activity that was 10 times or more on day 11 of differentiation induction, compared with that on day 0 of differentiation induction, were named “MAP-2-Luc/mouse ES cells.”

b) Differentiation into Nerve Cells

The MAP-2-Luc/mouse ES cells were maintained in Glasgow's MEM medium (G-MEM, produced by Thermo Fisher Scientific, catalog number: 11710035) containing 10% KnockOut serum replacement (KSR, produced by Thermo Fisher Scientific, catalog number: 10828028), 1% fetal bovine serum, 0.1 mM non-essential amino acid, 1 mM sodium pyruvate, 0.1 mM 2-mercaptoethanol, 2 U/ml leukemia inhibitory factor (produced by Oriental Yeast Co., Ltd., catalog number: NIB 47081000), and 100 μg/ml G418.

Differentiation into nerve cells was performed as follows. Specifically, the MAP-2-Luc/mouse ES cells were suspended in G-MEM medium containing 10% KSR, 2 mM glutamine, 0.1 mM non-essential amino acid, 1 mM sodium pyruvate, 0.1 mM 2-mercaptoethanol, and 1 μM SB431542 (produced by Sigma-Aldrich, catalog number: S4317), seeded in a Nunclon sphera 96U-well plate (Thermo Fisher Scientific, catalog number: 174929) at 6000 cells/well, and cultured for 6 days. The medium was then replaced with DMEM/F12 medium (referred to below as “medium for differentiation”) containing GlutaMax and N2 supplement (produced by Fujifilm Wako Pure Chemical Corporation, catalog number: 141-08941). The cells were cultured under these conditions until day 19 of differentiation to achieve neural differentiation.

On day 19 of differentiation induction, the cell mass was dispersed using a neuronal dispersion (produced by DS Pharma Biomedical Co., Ltd., catalog number: SBMBX9901D-2A), and suspended in neuronal medium (neurobasal medium containing 200 mM 1% glutamic acid and 2% B27). Then, 6.4×10⁴ cells were seeded on a 96-well white poly-D-lysine-coated plate with clear bottom (produced by Falcon BD, catalog number: 356651) and cultured.

### c) Measurement of Inhibitory Effect of Each Extract on Aβ₁₋₄₂-Induced Decrease in MAP-2 Promoter Activity

Various Kampo extracts were used as crude drug extracts (a kanbakutaisoto extract (produced by Matsuura Yakugyo Co., Ltd., production number: T-1808), a kanzokankyoto extract (produced by Matsuura Yakugyo Co., Ltd., production number: T-1808), a kikyoto extract (produced by Matsuura Yakugyo Co., Ltd., production number: T-1808), a kanzoto extract (produced by Matsuura Yakugyo Co., Ltd., production number: T-1808), and a Ginkgo biloba leaf extract (produced by Herb Green Health Biotech Co., Ltd.) to measure the inhibitory effect of each extract on the AB-induced decrease in MAP-2 promoter activity. Table 1 shows the types and mass ratios of the crude drug raw materials of each Kampo extract. Specifically, the measurement was performed as follows.

TABLE 1

| | Crude drug raw material |
|---|---|
| | (the numerical figures represent the |
| Kampo extract | mass ratio in each Kampo extract) |
| Kanzokankyoto | Glycyrrhiza: 4; processed ginger: 2 |
| Kanbakutaisoto | Glycyrrhiza: 5; jujube: 6; wheat: 20 |
| Kikyoto | Glycyrrhiza: 3; platycodon root: 2 |
| Kanzoto | Glycyrrhiza: 6 |

Dry human Aβ₁₋₄₂ (produced by Peptide Institute Inc., catalog number: 4349-v) was added and dissolved in 1,1,1,3,3,3-hexafluoro-2-propanol (HFIP). After being allowed to stand at room temperature for 16 hours, the resulting product was vacuum-dried for 2 hours and stored at −20° C. DMSO was added to the dry Aβ₁₋₄₂ treated with HFIP so that the concentration was 100 PM.

On day 9 of culturing the neuronally differentiated MAP-2-Luc/mouse ES cells in the neuronal medium, 100 μM Aβ₁₋₄₂ was added so that the final concentration of Aβ₁₋₄₂ was 10 μM. Next, 5 different types of Kampo extracts were separately added as samples to the medium and cultured for 5 days. In each medium, the concentrations of the kanbakutaisoto extract, the kanzokankyoto extract, the kikyoto extract, the kanzoto extract, and the Ginkgo biloba leave extract were 750 μg/ml, 75 μg/ml, 75 μg/ml, 25 μg/ml, and 250 μg/ml, respectively. Then, the luciferase activity was measured using a Triple Luciferase Assay System. The inhibition percentage for the Aβ₁₋₄₂-induced decrease in MAP-2 promoter activity was calculated as follows.

Inhibition percentage=(1−(X₀−Y_{Aβ})/(X₀−X_{Aβ}))×100 Equation 1

X₀: Fluorescence value of the solvent in the absence of Aβ
X_{Aβ}: Fluorescence value of the solvent in the presence of Aβ
Y_{Aβ}: Fluorescence value of the sample in the presence of Aβ

As shown in Table 2, the results reveal that the Kampo extracts that contained a glycyrrhiza extract (the kanzokankyoto extract, the kanbakutaisoto extract, the kikyoto extract, and the kanzoto extract) showed an inhibitory effect on the Aβ₁₋₄₂-induced decrease in MAP-2 promoter activity. Of the above Kampo extracts, the kanzokankyoto extract and the kanbakutaisoto extract (in particular, the kanzokankyoto extract) showed a high inhibitory effect.

TABLE 2

| | Inhibition |
|---|---|
| Extract | percentage |
| Kanzokankyoto | AA |
| Kanbakutaisoto | A |
| Kikyoto | B |
| Kanzoto | B |
| Ginkgo biloba leaf | C |

C: less than 10%
B: 10% to less than 30%
A: 30% to less than 50%
AA: 50% or more

### Example 2: Measurement of Inhibitory Effect on the Formation of Aβ Oligomers

Ninety microliters of a 5 μM solution obtained by adding DMSO to the HFIP-treated dry Aβ₁₋₄₂ so that the concentration was 500 μM, followed by dilution with PBS, was added to each well of a 96-well black plate (Greiner Bio-One International, catalog number: 655900). As a control, 90 μl of PBS containing 1% DMSO was added to each well. Next, 5 different types of Kampo extracts were separately added as samples in each amount of 10 μl and mixed with the medium, followed by allowing to stand in an incubator at 37° C. for 5 days. In each medium, the concentrations of the kanbakutaisoto extract, the kanzokankyoto extract, and the kanzoto extract were 750 μg/ml, 75 μg/ml, and 25 μg/ml, respectively. Ten microliters of each reaction solution was added to 100 μl of a thioflavin T solution (10 mM phosphate buffer (pH of 7.5), 5 μM thioflavin T, 100 mM NaCl) and mixed at room temperature for 30 minutes to measure the level of the formation of Aβ₁₋₄₂ oligomers by fluorescence measurement (Ex=435 nm, Em=485 nm). The inhibition percentage for the formation of Aβ oligomers was calculated as follows.

Inhibition percentage=(1−(Y_{Aβ}−Y₀)/((X_{Aβ}−X₀))×100 Equation 2

X_{Aβ}: Fluorescence value of the solvent in the presence of Aβ₁₋₄₂
X₀: Fluorescence value of the solvent in the absence of Aβ₁₋₄₂
Y_{Aβ}: Fluorescence value of the sample in the presence of Aβ₁₋₄₂
Y₀: Fluorescence value of the sample in the absence of Aβ₁₋₄₂

As shown in Table 3, the results reveal that the kanbakutaisoto extract, kanzokankyoto extract, and kanzoto extract showed a high inhibitory effect on the formation of Aβ oligomers.

TABLE 3

| | Inhibition |
|---|---|
| Extract | percentage |
| Kanzokankyoto | A |
| Kanbakutaisoto | A |
| Kanzoto | A |

C: less than 40%
B: 40% to less than 80%
A: 80% or more

The above results of Example 1 and Example 2 reveal that the Kampo extracts that contained a glycyrrhiza extract are capable of inhibiting the formation of Aβ oligomers and inhibiting nerve cell injury caused by Aβ.

### Sequence Listing

P20-165WO_PCT_Aβ [symbol] _20200805_174004_5.txt

1. A composition for inhibiting nerve cell injury caused by Aβ, comprising a Kampo extract that contains a glycyrrhiza extract.

2. The composition according to claim 1, wherein the Kampo extract does not contain a crude drug extract other than a glycyrrhiza extract, or further contains at least one crude drug extract selected from the group consisting of a processed ginger extract, a jujube extract, a wheat extract, a ginger extract, and a platycodon root extract.

3. The composition according to claim 1, wherein the Kampo extract further contains at least one crude drug extract selected from the group consisting of a processed ginger extract, a jujube extract, and a wheat extract.

4. The composition according to claim 1, wherein the Kampo extract is at least one member selected from the group consisting of a kanzokankyoto extract and a kanbakutaisoto extract.

5. The composition according to claim 4, wherein the mass ratio of crude drug raw materials for use in obtaining the kanzokankyoto extract is such that glycyrrhiza to processed ginger is 1:0.1 to 1.5.

6. The composition according to claim 4, wherein the mass ratio of crude drug raw materials for use in obtaining the kanzokankyoto extract is such that glycyrrhiza to processed ginger is 1:0.2 to 1.0.

7. The composition according to claim 4, wherein the mass ratio of crude drug raw materials for use in obtaining the kanbakutaisoto extract is such that glycyrrhiza to jujube to wheat is 1:0.5 to 2.0:2.6 to 6.6.

8. The composition according to claim 4, wherein the mass ratio of crude drug raw materials for use in obtaining the kanbakutaisoto extract is such that glycyrrhiza to jujube to wheat is 1:0.7 to 1.7:3.5 to 4.5.

9. The composition according to claim 1, which comprises the Kampo extract in an amount such that the daily application amount is 0.1 to 15 g.

10. The composition according to claim 1, which comprises the Kampo extract in an amount such that the daily application amount is 0.2 to 4.5 g.

11. The composition according to claim 1 for use in the prevention, amelioration, or treatment of diseases or symptoms in which nerve cell injury caused by Aβ is observed.

12. The composition according to claim 11, wherein the disease or symptom is Alzheimer's disease, mild cognitive impairment, Lewy body dementia, Down syndrome, Dutch hereditary cerebral hemorrhage with amyloidosis, Guam parkinsonism-dementia complex, cerebral amyloid angiopathy, inclusion body myositis, frontotemporal dementia, age-related macular degeneration, or Pick's disease.

13. The composition according to claim 1, which is a food composition.

14. A composition comprising a Kampo extract that contains a glycyrrhiza extract, for use in a method for inhibiting nerve cell injury caused by Aβ.

15. A method for inhibiting nerve cell injury caused by Aβ, comprising applying (e.g., ingesting, inoculating, or administering) a composition comprising a Kampo extract that contains a glycyrrhiza extract to a subject in need of inhibiting nerve cell injury caused by Aβ.

16. Use of a Kampo extract that contains a glycyrrhiza extract, in the manufacture of a composition for inhibiting nerve cell injury caused by Aβ.

17. Use of a composition comprising a Kampo extract that contains a glycyrrhiza extract, for inhibiting nerve cell injury caused by Aβ.
